(19) United States
(12) Patent Application Publication
(10) Pub. No.: US 2026/0100286 A1
(43) Pub. Date: Apr. 9, 2026

(54) MACHINE LEARNING BASED SMART ARRAY FOR HEALTHCARE PROVIDERS

(71) Applicant: Cerner Innovation, Inc., Kansas City, MO (US)

(72) Inventors: Praveen Bhat GURPUR, Bengaluru (IN); Suchitra Joyce PHILLIPS, Bangalore (IN)

(21) Appl. No.: 19/211,431

(22) Filed: May 19, 2025

Related U.S. Application Data
(60) Provisional application No. 63/705,074, filed on Oct. 9, 2024.

(51) Int. Cl. G16H 50/70; G16H 20/10; G16H 40/20; H04W 12/037

(52) U.S. Cl. CPC G16H 50/70; G16H 20/10; G16H 40/20; H04W 12/037

(57) ABSTRACT

Embodiments provide healthcare information to a plurality of healthcare providers. Embodiments receive first historical information corresponding to the plurality of healthcare providers and receive second historical information corresponding to external health care information sources. Embodiments train an artificial intelligence (“AI”) model using the first historical information and the second historical information. Embodiments receive current information corresponding to the plurality of healthcare providers and/or the external health care information sources. In response to the current information, embodiments generate one or more healthcare suggestions by the AI model and deliver the suggestions to one or more of the plurality of healthcare providers.

### CROSS REFERENCE TO RELATED APPLICATIONS

This application claims priority to U.S. Provisional Patent Application Ser. No. 63/705,074 filed on Oct. 9, 2024, the disclosure of which is hereby incorporated by reference.

### FIELD

One embodiment is directed generally to a computer system, and in particular to a computer system that implements machine learning to assist healthcare providers.

### BACKGROUND INFORMATION

In the United States, approximately 19% of the population lives in rural areas (i.e., approximately 1 in every 5 Americans). The residents of such areas seek medical help from physicians who live in these areas by visiting local clinics or small nursing homes. There are multiple challenges faced by physicians in rural areas including, limited access to healthcare services resulting in delayed care for patients, workforce shortages, professional isolation, limited diagnosis and treatment options, lower economic returns, technological disparities and limited educational and training opportunities such as Continuing Medical Education (“CME”). These challenges faced by rural physicians may reduce the quality of care delivered, resulting in poorer patient outcomes. This leads to many patients needing to travel to distant cities to seek better care, which in turn causes additional financial burden on patients, increased morbidity and mortality.

### SUMMARY

Embodiments provide healthcare information to a plurality of healthcare providers. Embodiments receive first historical information corresponding to the plurality of healthcare providers and receive second historical information corresponding to external health care information sources. Embodiments train an artificial intelligence (“AI”) model using the first historical information and the second historical information. Embodiments receive current information corresponding to the plurality of healthcare providers and/or the external health care information sources. In response to the current information, embodiments generate one or more healthcare suggestions by the AI model and deliver the suggestions to one or more of the plurality of healthcare providers.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIG. 1 illustrates an example of a system that includes a healthcare smart array system in accordance to embodiments.

FIG. 2 is a block diagram of the healthcare smart array system of FIG. 1 in the form of a computer server/system in accordance to an embodiment of the present invention.

FIG. 3 is a overview diagram of a healthcare smart array system interacting with rural or otherwise remote healthcare facilities in accordance to embodiments.

FIG. 4 is a flow/block diagram of the functionality of the healthcare smart array system of FIG. 1 when gathering and analyzing medical information in accordance to embodiments.

FIG. 5 illustrates various example parameters that are analyzed by the system as input data, and the resulting example insights/suggestions generated by the system as output data, in accordance to embodiment.

FIG. 6 is a flow diagram of functionality performed by rural hospitals, the cloud repository/system, and the AI algorithm that is part of the system in accordance to embodiments.

FIGS. 7-10 illustrate an example cloud infrastructure that can implement the healthcare smart array system of FIG. 1 in accordance to embodiments.

### DETAILED DESCRIPTION

One embodiments is an artificial intelligence (“AI”)/machine learning (“ML”) based system/array that includes a cloud repository of information collected from rural or otherwise remote physicians and that is combined with information collected from external sources to automatically generate AI based suggestions/insights that benefit subscribers of the system (i.e., physicians) in multiple ways, resulting in better healthcare delivery and patient outcomes.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

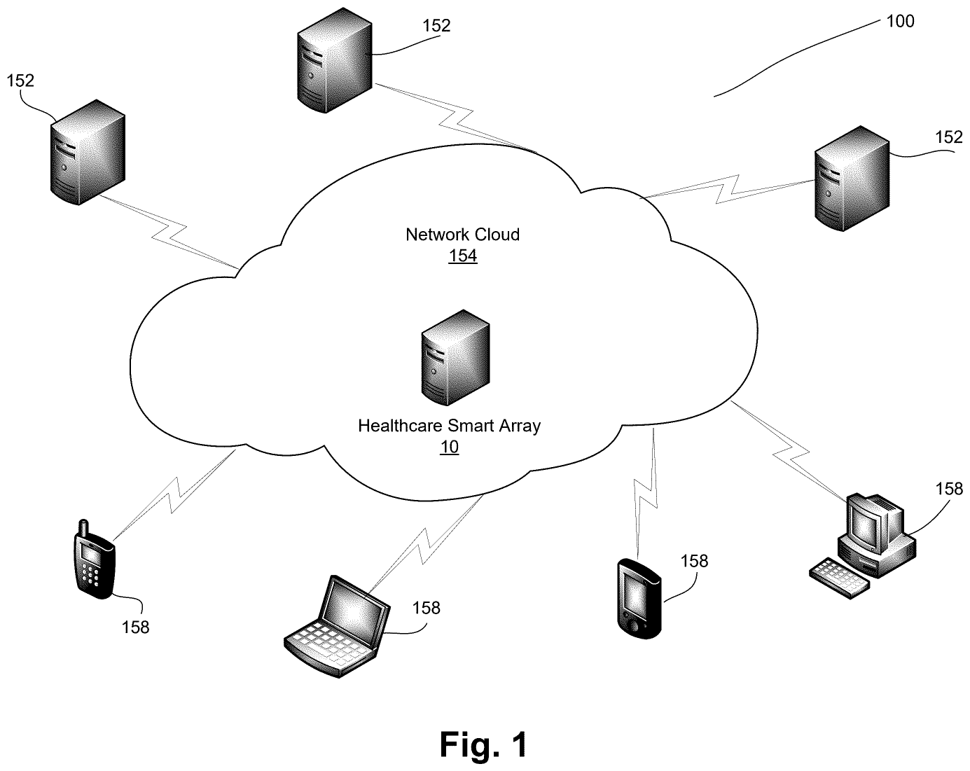

FIG. 1 illustrates an example of a system 100 that includes a healthcare smart array system 10 in accordance to embodiments. Healthcare smart array system 10 may be implemented within a computing environment that includes a communication network/cloud 154. Network 154 may be a private network that can communicate with a public network (e.g., the Internet) to access additional services 152 provided by a cloud services provider. Examples of communication networks include a mobile network, a wireless network, a cellular network, a local area network (“LAN”), a wide area network (“WAN”), other wireless communication networks, or combinations of these and other networks. Network cloud 154 may be administered by a service provider, such as via the Oracle Cloud Infrastructure (“OCI”) from Oracle Corp.

Tenants of the cloud services provider can be companies or any type of organization or groups whose members include users of services offered by the service provider. Services may include or be provided as access to, without limitation, an application, a resource, a file, a document, data, media, or combinations thereof. Users may have individual accounts with the service provider and organizations may have enterprise accounts with the service provider, where an enterprise account encompasses or aggregates a number of individual user accounts.

System 100 further includes client devices 158, which can be any type of device that can access network 154 and can obtain the benefits of the functionality of healthcare smart array system 10 of automatically generating and providing healthcare information and suggestions to remove providers. As disclosed herein, a “client” (also disclosed as a “client system” or a “client device”) may be a device or an application executing on a device. System 100 includes a number of different types of client devices 158 that each is able to communicate with network 154.

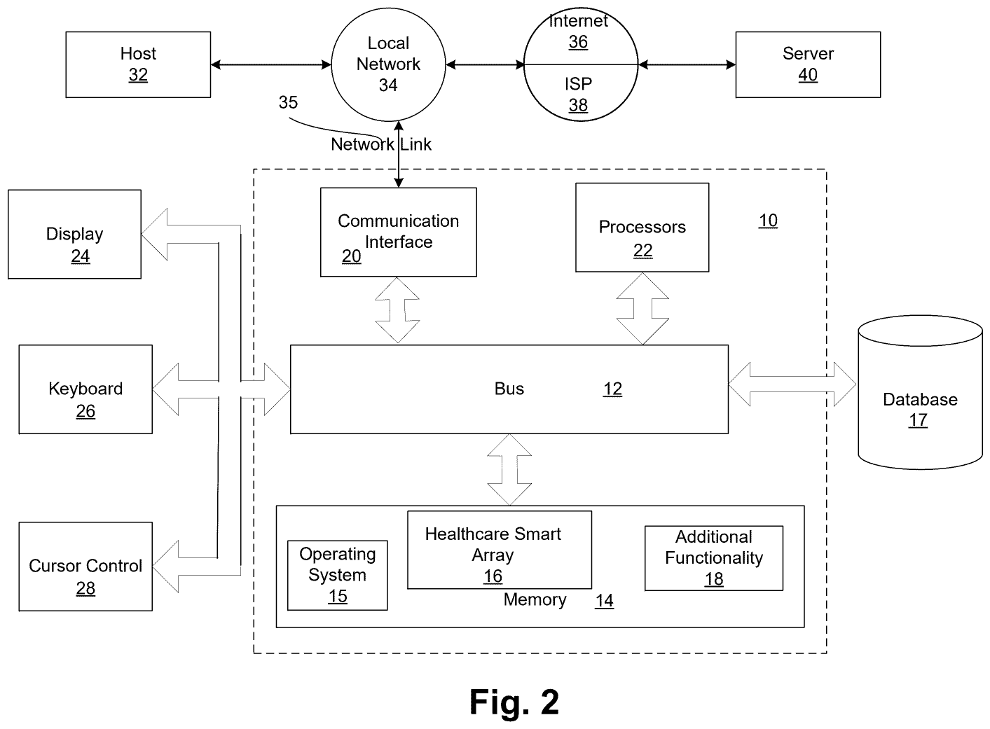

FIG. 2 is a block diagram of healthcare smart array system 10 of FIG. 1 in the form of a computer server/system 10 in accordance to an embodiment of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. One or more components of FIG. 2 can also be used to implement any of the elements of FIG. 1.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media, including transitory and non-transitory computer readable media. System 10 further includes a communication interface 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”). A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a healthcare smart array module 16 that provides medical information and suggestions using AI/ML, and all other functionality disclosed herein. System 10 can be part of a larger system. Therefore, system 10 can include one or more additional functional modules 18, such as an electronic medical records (“EMR”) or electronic health record (“EHR”) integrated solution (e.g., Oracle Health EHR from Oracle Corp.). A file storage device or database 17 is coupled to bus 12 to provide centralized storage for modules 16 and 18, including patient data, historical procedures, physician records, etc. In one embodiment, database 17 is a relational database management system (“RDBMS”) that can use Structured Query Language (“SQL”) to manage the stored data.

In embodiments, communication interface 20 provides a two-way data communication coupling to a network link 35 that is connected to a local network 34. For example, communication interface 20 may be an integrated services digital network (“ISDN”) card, cable modem, satellite modem, or a modem to provide a data communication connection to a corresponding type of telephone line or Ethernet. As another example, communication interface 20 may be a local area network (“LAN”) card to provide a data communication connection to a compatible LAN. Wireless links may also be implemented. In any such implementation, communication interface 20 sends and receives electrical, electromagnetic or optical signals that carry digital data streams representing various types of information.

Network link 35 typically provides data communication through one or more networks to other data devices. For example, network link 35 may provide a connection through local network 34 to a host computer 32 or to data equipment operated by an Internet Service Provider (“ISP”) 38. ISP 38 in turn provides data communication services through the Internet 36. Local network 34 and Internet 36 both use electrical, electromagnetic or optical signals that carry digital data streams. The signals through the various networks and the signals on network link 35 and through communication interface 20, which carry the digital data to and from computer system 10, are example forms of transmission media.

System 10 can send messages and receive data, including program code, through the network(s), network link 35 and communication interface 20. In the Internet example, a server 40 might transmit a requested code for an application program through Internet 36, ISP 38, local network 34 and communication interface 20. The received code may be executed by processor 22 as it is received, and/or stored in database 17, or other non-volatile storage for later execution.

In one embodiment, system 10 is a computing/data processing system including an application or collection of distributed applications for enterprise organizations, and may also implement logistics, manufacturing, and inventory management functionality. The applications and computing system 10 may be configured to operate locally or be implemented as a cloud-based networking system, for example in an infrastructure-as-a-service (“IAAS”), platform-as-a-service (“PAAS”), software-as-a-service (“SAAS”) architecture, or other type of computing solution.

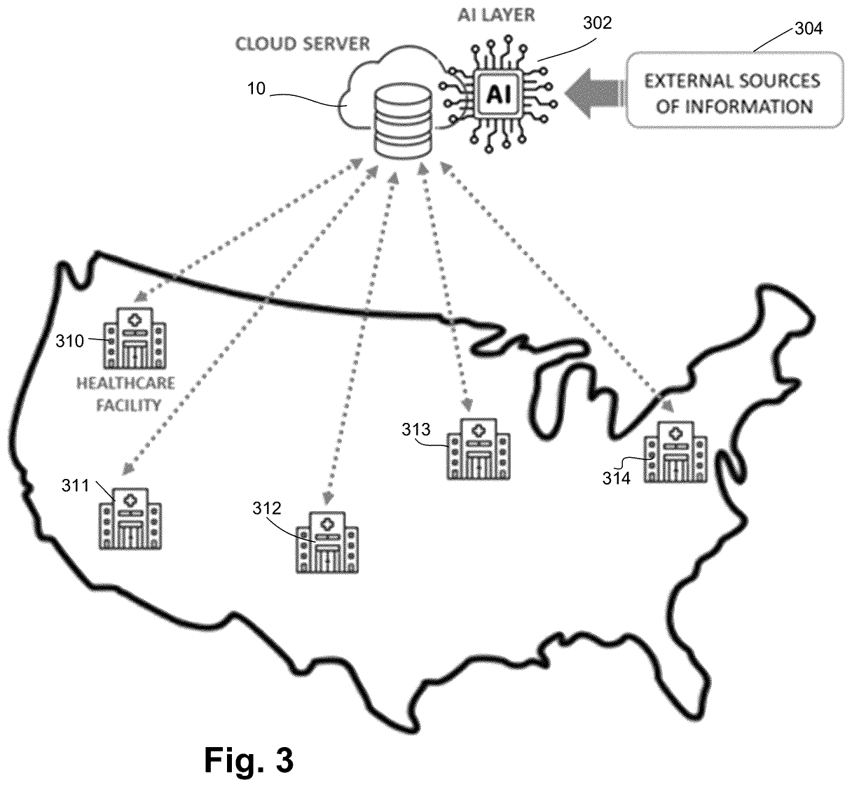

FIG. 3 is a overview diagram of healthcare smart array system 10 interacting with rural or otherwise remote healthcare facilities 310-314 in accordance to embodiments. An AI layer 302, which is integrated with system/server 10, extracts information from external sources 304 to be processed within server 10. Table 1 below provides examples of the data that is collected by system 10 from the respective healthcare facilities 310-314 and from external sources 304 in accordance to embodiments:

TABLE 1

| TYPE OF | | SOURCE OF |
|---|---|---|
| INFORMATION | DETAILS | INFORMATION |
| Healthcare facility | Location, type of facility (outpatient/hospital/nursing | Healthcare facility (310) |
| | home/day care center, etc.), any specialties available, | |
| | range of medical care/procedures /instruments/devices | |
| | available | |
| Healthcare personnel | Details of medical and paramedical staff of facility | Healthcare facility (310) |
| | including their education and experience, specialty, | |
| | contributions to medical field such as publications, | |
| | research work, etc. | |
| Patient demography | Age, gender, ethnicity, location | EMR system of the |
| | | healthcare facility (310) |
| Clinical information | Chief complaints, diagnosis in each visit, visit frequency, | EMR system of the |
| | presence of chronic conditions, healthcare conditions, | healthcare facility (310) |
| | medications being taken, procedures, diagnostic tests and | |
| | reports, physical activity levels, etc. | |
| Patient outcomes | Prognosis, follow-up, repeat visits, referrals, | EMR system of the |
| | admission/discharge if any, worsening, improvement, | healthcare facility (310) |
| | mortality | |
| Latest therapeutic | New generation medications, latest advancements in | American College of |
| protocols and drugs | clinical protocols, best clinical practices | Physicians |
| | | [acponline.org] (304) |
| | | American College of |
| | | Surgeons [facs.org] |
| | | (304) |
| | | UpToDate |
| | | [uptodate.com] (304) |
| Continuing Medical | CME events, workshops, seminars, symposia, trainings, | American Medical |
| Education | etc. | Association |
| (CME) opportunities | | [ama-assn.org] (304) |
| | | Accreditation Council |
| | | for Continuing Medical |
| | | Education [accme.org] |
| | | (304) |
| Local conditions | Weather, geography, terrain, water bodies, forests, | National Weather |
| | mountains, etc. | Service [weather.gov] |
| | | (304) |
| | | U.S Geological survey |
| | | [usgs.gov] (304) |
| Gatherings | Local/state/national festivals, religious/non-religious | Local news and |
| | gatherings, county fairs, carnivals, circus, exhibitions, | television channels (304) |
| | sports events, etc. | |
| Outbreaks/Accidents | Any local/state/national outbreaks of infectious | Centers for Disease |
| | diseases, food poisoning, industrial accidental spillage | Control [CDC.gov] (304) |
| | of toxins, fires, road traffic accidents, endemics, | World Health |
| | epidemics, pandemics | Organization [who.org] |
| | | (304) |
| | | Local news and |
| | | television channels (304) |
| Natural calamities | Forecasts and relay of information related to natural | National Weather |
| | disasters such as tornadoes, cyclones, earthquakes, | Service [weather.gov] |
| | tsunamis, forest fires, etc. | (304) |
| | | U.S Geological survey |
| | | [usgs.gov] (304) |
| | | Local news and |
| | | television channels (304) |

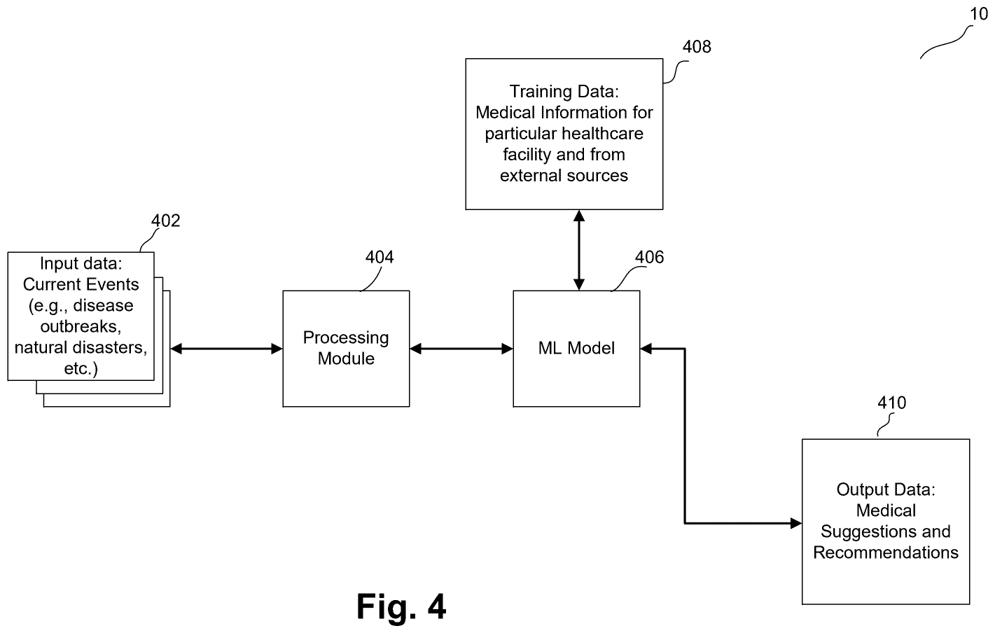

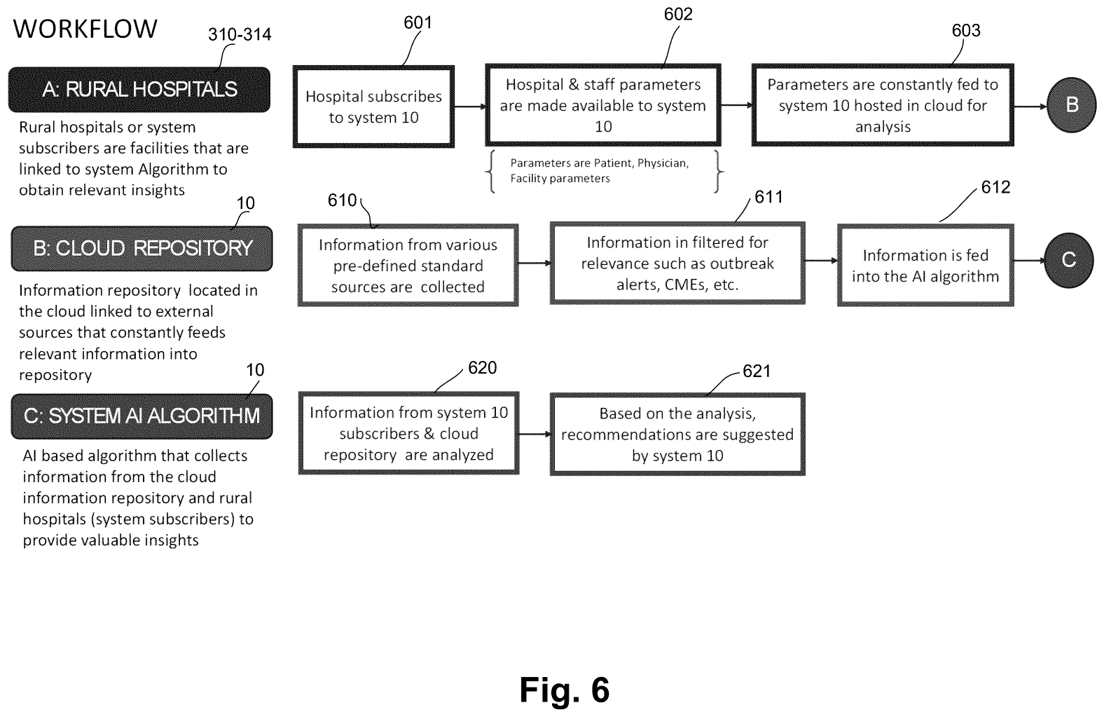

FIG. 4 is a flow/block diagram of the functionality of healthcare smart array system 10 of FIG. 1 when gathering and analyzing medical information in accordance to embodiments. In one embodiment, the functionality of the flow/block diagram of FIG. 4 (and FIG. 6 below) is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

System 10 includes input data 402, a processing module 404, a machine learning (“ML”) model/artificial intelligence (“AI”) model 306, training data 408, and output data 410. In general, system 10 is a trained system that gathers medical data, analyzes it, and generates outputs 410 in form of predictions, suggestions, curated information, useful pointers, etc.

Training data 408 allows model 406 to identify medically relevant information, and in general includes medical related concepts/information. Training data 408 enables system 10 to ingest two major categories of information (i.e., healthcare facility related information 310-314 and external sources of information 304) and perform analysis using AI technologies such as LLM or generative AI. The two categories of information it processes are:

1. Information related to the particular clinic/healthcare facility 310-314:
a. Data on the healthcare facility: Location, specialties, services, etc.
b. Information related to the healthcare personnel: Data on medical/paramedical staff, their education, experience, skills, contributions to medical field such as publications, etc.
c. Patient demography: Age, gender, ethnicity, address, etc., of patients that visit the facility.
d. Clinical information: Data from the EMR at the clinic is analyzed by system 10 comprehensively. This enables system 10 to get detailed information on patients including their conditions, clinical management, diagnostics tests, etc.
e. Patient outcomes: For each patient, system 10 analyzes their repeat visits, time intervals between visits, diagnosis in each visit, worsening/improvement, referrals, mortality, hospitalization data (if available), etc.

2. Information from external sources 304:
a. Latest therapeutic protocols and drugs: System 10 looks for information on new generation of medications, latest advances in clinical protocols and best clinical practices. It gathers this information automatically from websites such as American College of Physicians (acponline.org), American College of Surgeons (facs.org), UpToDate (uptodate.com), etc.
b. Opportunities for Continuing Medical Education (“CME”): System 10 looks for information on CME events, workshops, seminars, symposia, conferences, trainings etc. from American Medical Association (ama-assn.org), Accreditation Council for Continuing Medical Education (accme.org), etc.
c. Local conditions: Weather, geography, terrain, water bodies, forests, mountains, etc. from National Weather Service (weather.gov), U.S. Geological Survey (usgs.gov), etc.
d. Local gatherings: Local/state/national festivals, religious/non-religious gatherings, county fairs, carnivals, circus, exhibitions, sports events, etc. from local news and television channels.
e. Disease outbreaks or accidents: Any local/state/national outbreaks of infectious diseases, food poisoning, industrial accidental spillage of toxins, fires, road traffic accidents, endemics, epidemics, pandemics from Centers for Disease Control (CDC.gov), World Health Organization (who.org), local news and television channels, etc.
f. Natural disasters or calamities: Forecasts and relay of information related to natural disasters such as tornadoes, cyclones, earthquakes, tsunamis, forest fires, etc. from National Weather Service (weather.gov), U.S. Geological survey (usgs.gov), Local news and television channels, etc.

Training data 408 may be labeled data. Processing module 404 can be used to process input data 402 so that it can be used/comprehended by model 406 (e.g., current event information, unstructured data, etc.).

ML model 406 can be any type of machine learning model (e.g., generative model, neural network, deep learning, NLP, support vector machine (“SVM”), random forests, gradient boosting, large language model (“LLM”) etc.) that is trained by training data 408. In one embodiment, ML model 406 implements generative AI. Generative AI refers to artificial intelligence systems that can create new content, such as text, images, music, and other media, by learning from existing data. Unlike traditional AI, which typically focuses on recognizing patterns and making predictions based on input data, generative AI models are designed to generate new, original outputs that are similar to the data they were trained on. ML model 406 generates output data 410, which includes medically useful information to be delivered to medical facilities 310-314.

The content of input data 402 can include any of the historical information provided as training data 408, except that it is provided in a current timeframe (i.e., “live” data). The information of training data 408 is not static, but dynamic. In other words, the information contained in the above sources changes continuously, with some information such as weather changing every minute, whereas others such as new drugs changing every few weeks or months. System 10 is configured to automatically and continuously update itself on the above information, via input data 402 and/or training data 408, so that it remains current and updated at all times.

System 10 can calculate parameters related to patients in response to input data 402, such as the percentage of patients who had worsening symptoms and were referred, the percentage of patients who are visiting repeatedly for the same treatable conditions, the percentage of patients who are being referred, but could actually be treated in the facility itself, etc., and assesses if the above percentage numbers exceed a certain threshold. The threshold can either be set by the clinic or system 10 as per recommendations by national bodies who publish standards. If the percentage numbers exceed the threshold, system 10 automatically generates advice/suggestions to the physician at the respective facility 310-314.

System 10 can calculate parameters related to the physician in response to input data 402, such as the time elapsed since the last time the physician underwent a training/CME, or the percentage of patients who have a condition for which the physician has not undergone training/CME, or if the physician has recently been trained in emergency medical procedures, and assesses if the above parameters exceed an allowable threshold. The threshold can either be set by the clinic or system 10 as per recommendations by national bodies who publish standards. If the numbers exceed the threshold, system 10 automatically generates advice/suggestions to the physician at the respective facility 310-314.

System 10 constantly monitors, as input data 402, websites such as of the CDC and WHO, as well as local news and TV channels to look for any disease outbreaks, natural disasters, etc. in the local area. This is enabled by LLM technology where it looks for certain keywords occurring frequently, patterns in such words in radio/TV broadcasts, etc. It then assesses if the specific facility is prepared to handle the outbreak/disaster etc. As an example, if there is an outbreak of a disease such as influenza, embodiments can automatically check the hospital vaccine inventory to see if there are sufficient number of flu vaccine doses available, based on assessing how many people in the local population may need vaccines. If a sufficient number of vaccine doses are not available, embodiments can immediately issue an alert to the physician and supervisor of the respective facility to procure the doses.

As another example, if it is a natural disaster such as a forest fire, system 10 can automatically check the hospital pharmacy to see if sufficient amount of medicines to treat burns, smoke inhalation, dehydration, etc., are available, based on assessing how many people in the local population may be living in areas where they could get affected by the fire. If a sufficient number of medicine doses are not available, embodiments can immediately issue an alert to the physician and supervisor of the facility to procure the medications.

System 10 learns and becomes better iteratively, by analyzing the decisions taken by the physician/other human consumers of the insights outputted by system 10 and undergoing retraining as the additional training data becomes available. For example, assume system 10 recommends (as output data 410) procuring a certain number of flu vaccines, based on a flu outbreak. However, the physician orders 50% more vaccines than recommended by system 10, as she knew that there would be at least 100 workers arriving in the town starting the following week due to a major construction activity in this town. Therefore, the physician foresaw that she would be needing more vaccines than suggested by system 10. This enables system 10 to learn from this experience so that the next time, it needs to account for such variables that are unexpected but have a bearing on its outputs.

In connection with ML model 406, embodiments in general can utilize one or more machine learning models to analyze health data, such as data stored via a user's personal health record. A “machine learning model,” as used herein, refers to a construct that is configured (e.g., trained using training data) to make predictions, provide probabilities, augment data, and/or generate data. For example, training data for supervised learning can include items with various parameters and an assigned classification. A new data item can have parameters that a model can use to assign a classification to the new data item. Machine learning models can be configured for various situations, data types, sources, and output formats.

Training data can be any set of data capable of training machine learning models, such as a set of features with corresponding labels for supervised learning. Training data can be used to train machine learning models to generate trained machine learning models. For example, any suitable training technique (e.g., supervised training via gradient descent, unsupervised training, etc.) can be used to update a configuration of machine learning models (e.g., train the weights of a machine learning model) using training data.

The architecture of implemented machine learning models can include any suitable machine learning model components. For example, a neural network can be implemented along with a given cost function (e.g., for training/gradient calculation). The neural network can include any number of hidden layers (e.g., 0, 1, 2, 3, or many more), and can include feed forward neural networks, recurrent neural networks, convolution neural networks, transformer networks, encoder-decoder architectures, large language models, and any other suitable type. In some implementations, the neural network can be configured for deep learning, for example based on the number of hidden layers implemented.

In some implementations, machine learning models can be an ensemble learning model. Multiple models can be stacked, for example with the output of a first model feeding into the input of a second model. Some implementations can include a number of layers of prediction models. In some implementations, features utilized by machine learning models can also be determined, for example via any suitable feature engineering techniques.

In some implementations, the design of machine learning models can be tuned during training, retraining, and/or updated training. For example, tuning can include adjusting a number of hidden layers in a neural network, adjusting a kernel calculation used to implement a support vector machine, and the like. This tuning can also include adjusting/selecting features used by the machine learning models. Various tuning configurations (e.g., different versions of the machine learning model and features) can be implemented while training in order to arrive at a configuration for machine learning models that, when trained, achieves desired performance (e.g., performs predictions at a desired level of accuracy, run according to desired resource utilization/time metrics, and the like). Retraining and updating the training can include training with updated training data. For example, the training data can be updated to incorporate observed data, or data that has otherwise been labeled (e.g., for use with supervised learning).

Implementations can fine-tune large language models with domain specific language data. For example, historical health data can be aggregated to generate a set of training data specific to healthcare. A pre-trained large language model can be fine-tuned with the set of training data to generate a large language model configured for health data. For example, one or more layers, nodes, weights, etc. of the pre-trained large language model can be updated and/or added via the fine-tuning to configure the large language model for health data. In some implementations, the fine-tuned large language model can be prompted to analyze health data and return results (e.g., data visualizations, tables of data, answers to queries, etc.).

In some examples, embodiments can automatically flag conditions and/or suggest changes to the user's care plan based on the data being generated related to the care plan. For example, natural language processing models can process the user's patient notes, and certain sentiment can be mapped to predefined recommendations, such as reducing the intensity of physical therapy when the patient's reported pain is high, recommending a patient consultation when the patient notes indicate confusion with the care plan, triggering an alert that schedules a patient consultation when monitored health metric(s) fail to meet a criteria, and the like.

Machine learning models and/or artificial intelligence can be implemented to process the user's health data and/or the data being generated related to a care plan to flag conditions and/or suggest changes. For example, monitored metric(s) can be processed by the models, patient notes can be processed by the models, data generated via a user visit to a hospital/doctor's office can be processed by the models, and any other suitable user health data can be processed by the models. The models, based on the processing, can generate recommended changes to the care plan and/or raise flags for care team review.

In connection with general training, ML model 406 is trained in medical and clinical information using generally the same curriculum used for training human physicians. ML model 406 has the ability to search information on clinical medicine from sites such as AMA, CDC, WHO, etc., and from journal publications from PubMed, Scopus, Medline, EMBASE, Google Scholar, etc., to update itself on the latest developments on best protocols, latest pharmaceuticals, etc.

In connection with specific training for a given physician, ML model 406 is trained via the EMRs of all patients treated by a given physician to gather data using one or more of the following technologies (i.e., ML model can be implemented by one or more (via multiple models)) of the following:

ML model 406, for specific training for a given physician, may be implemented by a supervised learning algorithms such as decision trees, support vector machines, and k-nearest neighbors. These algorithms are used to find patterns in labeled data, such as diagnosis, medications ordered, lab orders, etc.

ML model 406, for specific training for a given physician, may be implemented by an unsupervised learning algorithm such as clustering (e.g., k-means, hierarchical clustering) and association rule learning. These algorithms help detect hidden patterns in unlabeled data by grouping similar items or finding relationships. The unlabeled data may include physician notes, discharge summary, patient admission data, patient feedback data etc.

ML model 406, for specific training for a given physician, may be implemented by a deep learning model. For example, convolutional neural networks (“CNN”s) can be used for image data, while recurrent neural networks (“RNN”s) and transformers are used for sequential or time-series data. The sequential or time-series data may include X Rays, CT Scans, MRI scans, ultrasound scans, continuous vitals, data from sensors, etc.

ML model 406, for specific training for a given physician, may be implemented by an autoencoder, which can be used for anomaly detection or for reducing dimensionality, which identifies patterns within high-dimensional data. The high-dimensional data may include infectious diseases, rare diseases, patient deterioration, genomic data, etc.

ML model 406 may be implemented by natural language processing model such as BERT, GPT, and other transformer-based architectures that help analyze textual data to identify trends, sentiment, and other linguistic patterns. The textual data may include clinical notes, EMR summarization, patient sentiment analysis, medical coding and billing, voice/text recognition from transcription, social determinants of health analysis, etc.

ML model 406 may be implemented by an association rule learning model. Techniques such as the Apriori and Eclat algorithms are used for finding interesting associations or correlations in transactional datasets, commonly applied in market basket analysis. The textual data may include medication interaction and prescription patterns, diagnosis concurrence analysis, treatment outcome analysis, patient risk factor analysis, lab test patterns/anomalies, chronic disease management, re-visit patterns, preventive health insights, social and behavioral health patterns, ER visits pattern, etc.

ML model 406 may be implemented by a reinforcement learning model. Reinforcement learning can help in discovering optimal patterns for sequential or time-based data where actions influence future data points. The sequential or time-based data may include personalized treatment, optimizing drug dosage, predicting and preventing patient deterioration, chronic disease management, optimizing rehabilitation protocols, ER triage and treatment, preventive health, etc.

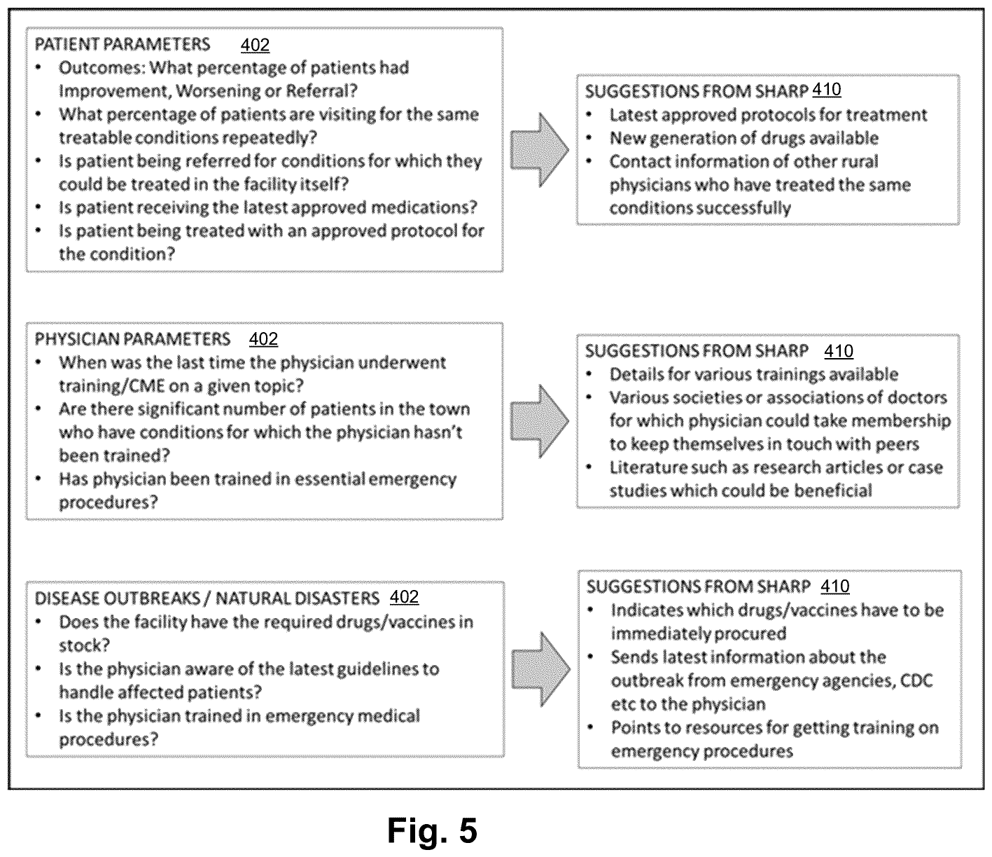

FIG. 5 illustrates various example parameters (shown on left) that are analyzed by system 10 as input data 402, and the resulting example insights/suggestions generated by system 10 (referred to as “smart healthcare array for rural physicians” (“SHARP”)) as output data 410, in accordance to embodiment.

FIG. 6 is a flow diagram of functionality performed by rural hospitals 310-314, cloud repository/system 10, and the AI algorithm 406 that is part of system 10 in accordance to embodiments.

At the rural hospitals 310-314, the hospital subscribes to system 10 (601); hospital and staff parameters (e.g., patient, physician and facility parameters) are made available to system 10 (602); and parameters are constantly fed to system 10 hosted in cloud 104 for analysis (603) as input data 402. Example of parameters are shown on the left side of FIG. 5.

At the cloud repository (i.e., system 10), information from various predefined standard sources are collected (610); the information in filtered for relevance such as outbreak alerts, CMEs, etc. (611); and the information (i.e., training data 408) is fed into the AI algorithm (i.e., the ML model 406) (612).

At the AI algorithm/ML model 406, information from system 10 subscribers (i.e., input data 402) and the cloud repository are analyzed (620); and based on the analysis, recommendations are suggested by system 10 (621) as output data 410. Example suggestions are shown on the right side of FIG. 5.

### Example Cloud Infrastructure

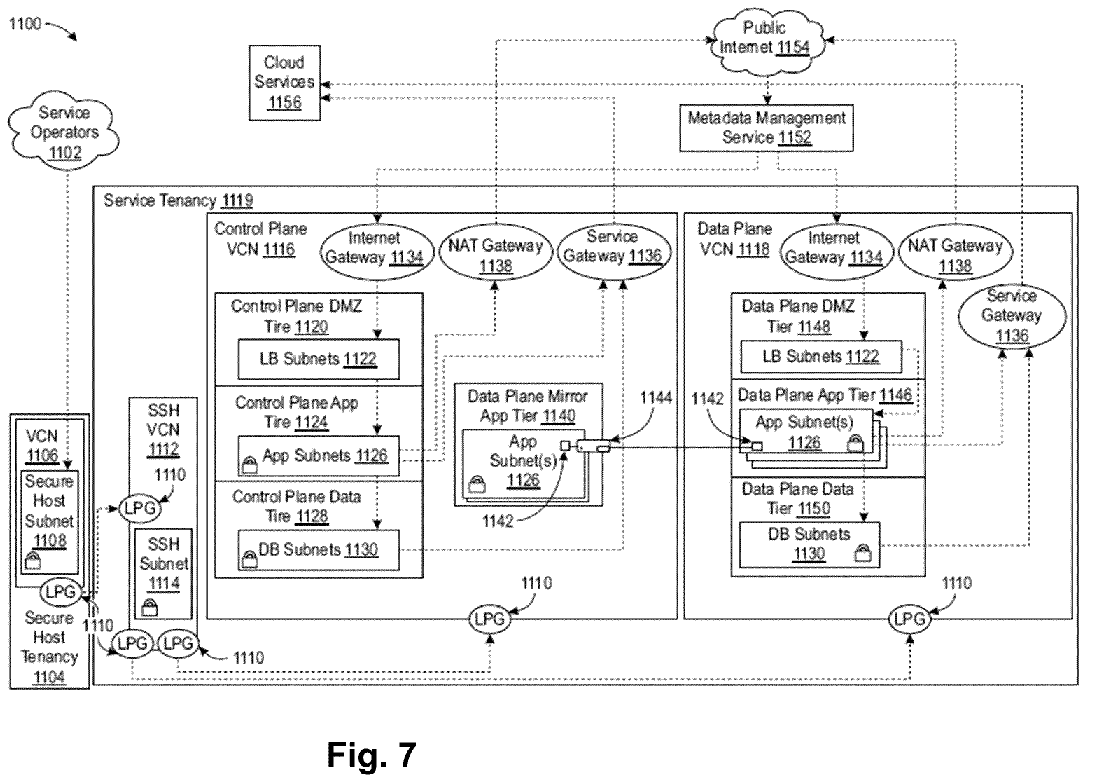

FIGS. 7-10 illustrate an example cloud infrastructure that can implement system 100 that can include healthcare smart array system 10 of FIG. 1 in accordance to embodiments. The use of the cloud infrastructure, as opposed to an on-premise implementation, allows for training data 408 to be receive from many different users that are interacting with the application of interest, which enhances the accuracy of ML model 406.

As disclosed above, infrastructure as a service (“IaaS”) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (e.g., billing, monitoring, logging, security, load balancing and clustering, etc.). Thus, as these services may be policy-driven, IaaS users may be able to implement policies to drive load balancing to maintain application availability and performance.

In some instances, IaaS customers may access resources and services through a wide area network (“WAN”), such as the Internet, and can use the cloud provider's services to install the remaining elements of an application stack. For example, the user can log in to the IaaS platform to create virtual machines (“VM”s), install operating systems (“OS”s) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different problems for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (“VPC”s) (e.g., a potentially on-demand pool of configurable and/or shared computing resources), also known as a core network. In some examples, there may also be one or more security group rules provisioned to define how the security of the network will be set up and one or more virtual machines. Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 7 is a block diagram 1100 illustrating an example pattern of an IaaS architecture, according to at least one embodiment. Service operators 1102 can be communicatively coupled to a secure host tenancy 1104 that can include a virtual cloud network (“VCN”) 1106 and a secure host subnet 1108. In some examples, the service operators 1102 may be using one or more client computing devices, which may be portable handheld devices (e.g., an iPhone®, cellular telephone, an iPad®, computing tablet, a personal digital assistant (“PDA”)) or wearable devices (e.g., a Meta Quest® head mounted display), running software such as Microsoft Windows Mobile®, and/or a variety of mobile operating systems such as iOS, Windows Phone, Android, BlackBerry 8, Palm OS, and the like, and being Internet, e-mail, short message service (“SMS”), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1106 and/or the Internet.

The VCN 1106 can include a local peering gateway (“LPG”) 1110 that can be communicatively coupled to a secure shell (“SSH”) VCN 1112 via an LPG 1110 contained in the SSH VCN 1112. The SSH VCN 1112 can include an SSH subnet 1114, and the SSH VCN 1112 can be communicatively coupled to a control plane VCN 1116 via the LPG 1110 contained in the control plane VCN 1116. Also, the SSH VCN 1112 can be communicatively coupled to a data plane VCN 1118 via an LPG 1110. The control plane VCN 1116 and the data plane VCN 1118 can be contained in a service tenancy 1119 that can be owned and/or operated by the IaaS provider.

The control plane VCN 1116 can include a control plane demilitarized zone (“DMZ”) tier 1120 that acts as a perimeter network (e.g., portions of a corporate network between the corporate intranet and external networks). The DMZ-based servers may have restricted responsibilities and help keep security breaches contained. Additionally, the DMZ tier 1120 can include one or more load balancer (“LB”) subnet(s) 1122, a control plane app tier 1124 that can include app subnet(s) 1126, a control plane data tier 1128 that can include database (DB) subnet(s) 1130 (e.g., frontend DB subnet(s) and/or backend DB subnet(s)). The LB subnet(s) 1122 contained in the control plane DMZ tier 1120 can be communicatively coupled to the app subnet(s) 1126 contained in the control plane app tier 1124 and an Internet gateway 1134 that can be contained in the control plane VCN 1116, and the app subnet(s) 1126 can be communicatively coupled to the DB subnet(s) 1130 contained in the control plane data tier 1128 and a service gateway 1136 and a network address translation (NAT) gateway 1138. The control plane VCN 1116 can include the service gateway 1136 and the NAT gateway 1138.

The control plane VCN 1116 can include a data plane mirror app tier 1140 that can include app subnet(s) 1126. The app subnet(s) 1126 contained in the data plane mirror app tier 1140 can include a virtual network interface controller (VNIC) 1142 that can execute a compute instance 1144. The compute instance 1144 can communicatively couple the app subnet(s) 1126 of the data plane mirror app tier 1140 to app subnet(s) 1126 that can be contained in a data plane app tier 1146.

The data plane VCN 1118 can include the data plane app tier 1146, a data plane DMZ tier 1148, and a data plane data tier 1150. The data plane DMZ tier 1148 can include LB subnet(s) 1122 that can be communicatively coupled to the app subnet(s) 1126 of the data plane app tier 1146 and the Internet gateway 1134 of the data plane VCN 1118. The app subnet(s) 1126 can be communicatively coupled to the service gateway 1136 of the data plane VCN 1118 and the NAT gateway 1138 of the data plane VCN 1118. The data plane data tier 1150 can also include the DB subnet(s) 1130 that can be communicatively coupled to the app subnet(s) 1126 of the data plane app tier 1146.

The Internet gateway 1134 of the control plane VCN 1116 and of the data plane VCN 1118 can be communicatively coupled to a metadata management service 1152 that can be communicatively coupled to public Internet 1154. Public Internet 1154 can be communicatively coupled to the NAT gateway 1138 of the control plane VCN 1116 and of the data plane VCN 1118. The service gateway 1136 of the control plane VCN 1116 and of the data plane VCN 1118 can be communicatively coupled to cloud services 1156.

In some examples, the service gateway 1136 of the control plane VCN 1116 or of the data plane VCN 1118 can make application programming interface (“API”) calls to cloud services 1156 without going through public Internet 1154. The API calls to cloud services 1156 from the service gateway 1136 can be one-way: the service gateway 1136 can make API calls to cloud services 1156, and cloud services 1156 can send requested data to the service gateway 1136. But, cloud services 1156 may not initiate API calls to the service gateway 1136.

In some examples, the secure host tenancy 1104 can be directly connected to the service tenancy 1119, which may be otherwise isolated. The secure host subnet 1108 can communicate with the SSH subnet 1114 through an LPG 1110 that may enable two-way communication over an otherwise isolated system. Connecting the secure host subnet 1108 to the SSH subnet 1114 may give the secure host subnet 1108 access to other entities within the service tenancy 1119.

The control plane VCN 1116 may allow users of the service tenancy 1119 to set up or otherwise provision desired resources. Desired resources provisioned in the control plane VCN 1116 may be deployed or otherwise used in the data plane VCN 1118. In some examples, the control plane VCN 1116 can be isolated from the data plane VCN 1118, and the data plane mirror app tier 1140 of the control plane VCN 1116 can communicate with the data plane app tier 1146 of the data plane VCN 1118 via VNICs 1142 that can be contained in the data plane mirror app tier 1140 and the data plane app tier 1146.

In some examples, users of the system, or customers, can make requests, for example create, read, update, or delete (“CRUD”) operations, through public Internet 1154 that can communicate the requests to the metadata management service 1152. The metadata management service 1152 can communicate the request to the control plane VCN 1116 through the Internet gateway 1134. The request can be received by the LB subnet(s) 1122 contained in the control plane DMZ tier 1120. The LB subnet(s) 1122 may determine that the request is valid, and in response to this determination, the LB subnet(s) 1122 can transmit the request to app subnet(s) 1126 contained in the control plane app tier 1124. If the request is validated and requires a call to public Internet 1154, the call to public Internet 1154 may be transmitted to the NAT gateway 1138 that can make the call to public Internet 1154. Memory that may be desired to be stored by the request can be stored in the DB subnet(s) 1130.

In some examples, the data plane mirror app tier 1140 can facilitate direct communication between the control plane VCN 1116 and the data plane VCN 1118. For example, changes, updates, or other suitable modifications to configuration may be desired to be applied to the resources contained in the data plane VCN 1118. Via a VNIC 1142, the control plane VCN 1116 can directly communicate with, and can thereby execute the changes, updates, or other suitable modifications to configuration to, resources contained in the data plane VCN 1118.

In some embodiments, the control plane VCN 1116 and the data plane VCN 1118 can be contained in the service tenancy 1119. In this case, the user, or the customer, of the system may not own or operate either the control plane VCN 1116 or the data plane VCN 1118. Instead, the IaaS provider may own or operate the control plane VCN 1116 and the data plane VCN 1118, both of which may be contained in the service tenancy 1119. This embodiment can enable isolation of networks that may prevent users or customers from interacting with other users', or other customers', resources. Also, this embodiment may allow users or customers of the system to store databases privately without needing to rely on public Internet 1154, which may not have a desired level of security, for storage.

In other embodiments, the LB subnet(s) 1122 contained in the control plane VCN 1116 can be configured to receive a signal from the service gateway 1136. In this embodiment, the control plane VCN 1116 and the data plane VCN 1118 may be configured to be called by a customer of the IaaS provider without calling public Internet 1154. Customers of the IaaS provider may desire this embodiment since database(s) that the customers use may be controlled by the IaaS provider and may be stored on the service tenancy 1119, which may be isolated from public Internet 1154.

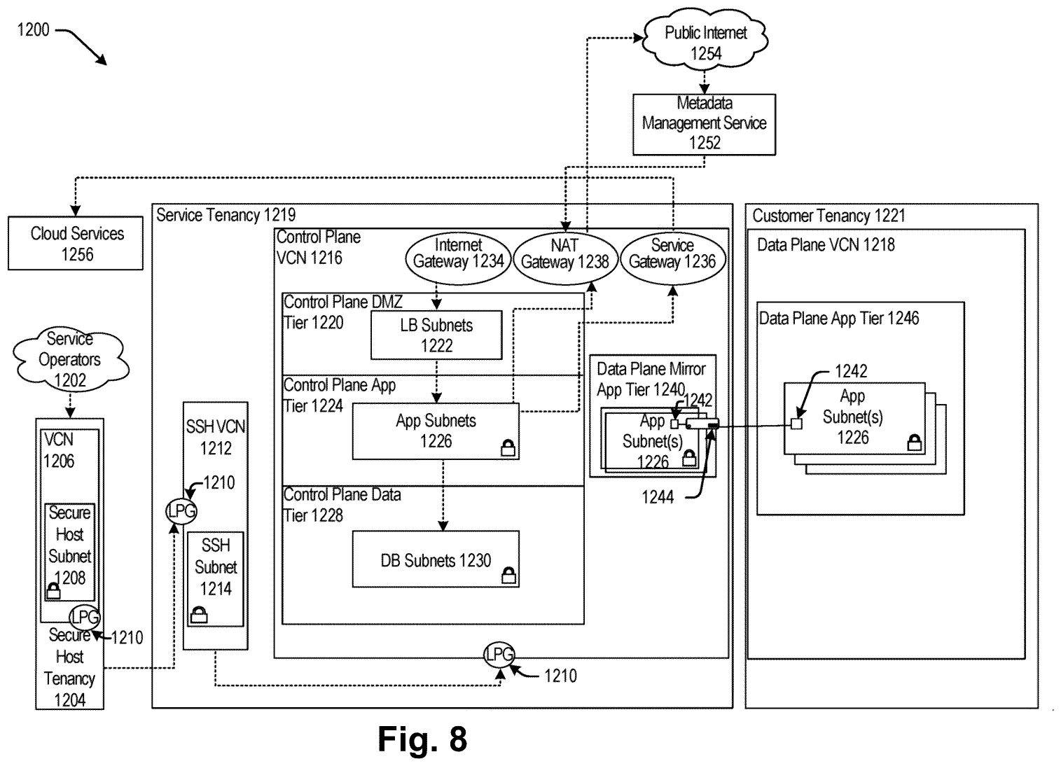

FIG. 8 is a block diagram 1200 illustrating another example pattern of an IaaS architecture, according to at least one embodiment. Service operators 1202 (e.g. service operators 1102) can be communicatively coupled to a secure host tenancy 1204 (e.g. the secure host tenancy 1104) that can include a virtual cloud network (VCN) 1206 (e.g. the VCN 1106) and a secure host subnet 1208 (e.g. the secure host subnet 1108). The VCN 1206 can include a local peering gateway (LPG) 1210 (e.g. the LPG 1110) that can be communicatively coupled to a secure shell (SSH) VCN 1212 (e.g. the SSH VCN 1112 10) via an LPG 1110 contained in the SSH VCN 1212. The SSH VCN 1212 can include an SSH subnet 1214 (e.g. the SSH subnet 1114), and the SSH VCN 1212 can be communicatively coupled to a control plane VCN 1216 (e.g. the control plane VCN 1116) via an LPG 1210 contained in the control plane VCN 1216. The control plane VCN 1216 can be contained in a service tenancy 1219 (e.g. the service tenancy 1119), and the data plane VCN 1218 (e.g. the data plane VCN 1118) can be contained in a customer tenancy 1221 that may be owned or operated by users, or customers, of the system.

The control plane VCN 1216 can include a control plane DMZ tier 1220 (e.g. the control plane DMZ tier 1120) that can include LB subnet(s) 1222 (e.g. LB subnet(s) 1122), a control plane app tier 1224 (e.g. the control plane app tier 1124) that can include app subnet(s) 1226 (e.g. app subnet(s) 1126), a control plane data tier 1228 (e.g. the control plane data tier 1128) that can include database (DB) subnet(s) 1230 (e.g. similar to DB subnet(s) 1130). The LB subnet(s) 1222 contained in the control plane DMZ tier 1220 can be communicatively coupled to the app subnet(s) 1226 contained in the control plane app tier 1224 and an Internet gateway 1234 (e.g. the Internet gateway 1134) that can be contained in the control plane VCN 1216, and the app subnet(s) 1226 can be communicatively coupled to the DB subnet(s) 1230 contained in the control plane data tier 1228 and a service gateway 1236 and a network address translation (NAT) gateway 1238 (e.g. the NAT gateway 1138). The control plane VCN 1216 can include the service gateway 1236 and the NAT gateway 1238.

The control plane VCN 1216 can include a data plane mirror app tier 1240 (e.g. the data plane mirror app tier 1140) that can include app subnet(s) 1226. The app subnet(s) 1226 contained in the data plane mirror app tier 1240 can include a virtual network interface controller (VNIC) 1242 (e.g. the VNIC of 1142) that can execute a compute instance 1244 (e.g. similar to the compute instance 1144). The compute instance 1244 can facilitate communication between the app subnet(s) 1226 of the data plane mirror app tier 1240 and the app subnet(s) 1226 that can be contained in a data plane app tier 1246 (e.g. the data plane app tier 1146) via the VNIC 1242 contained in the data plane mirror app tier 1240 and the VNIC 1242 contained in the data plane app tier 1246.

The Internet gateway 1234 contained in the control plane VCN 1216 can be communicatively coupled to a metadata management service 1252 (e.g. the metadata management service 1152) that can be communicatively coupled to public Internet 1254 (e.g. public Internet 1154). Public Internet 1254 can be communicatively coupled to the NAT gateway 1238 contained in the control plane VCN 1216. The service gateway 1236 contained in the control plane VCN 1216 can be communicatively couple to cloud services 1256 (e.g. cloud services 1156).

In some examples, the data plane VCN 1218 can be contained in the customer tenancy 1221. In this case, the IaaS provider may provide the control plane VCN 1216 for each customer, and the IaaS provider may, for each customer, set up a unique compute instance 1244 that is contained in the service tenancy 1219. Each compute instance 1244 may allow communication between the control plane VCN 1216, contained in the service tenancy 1219, and the data plane VCN 1218 that is contained in the customer tenancy 1221. The compute instance 1244 may allow resources that are provisioned in the control plane VCN 1216 that is contained in the service tenancy 1219, to be deployed or otherwise used in the data plane VCN 1218 that is contained in the customer tenancy 1221.

In other examples, the customer of the IaaS provider may have databases that live in the customer tenancy 1221. In this example, the control plane VCN 1216 can include the data plane mirror app tier 1240 that can include app subnet(s) 1226. The data plane mirror app tier 1240 can reside in the data plane VCN 1218, but the data plane mirror app tier 1240 may not live in the data plane VCN 1218. That is, the data plane mirror app tier 1240 may have access to the customer tenancy 1221, but the data plane mirror app tier 1240 may not exist in the data plane VCN 1218 or be owned or operated by the customer of the IaaS provider. The data plane mirror app tier 1240 may be configured to make calls to the data plane VCN 1218, but may not be configured to make calls to any entity contained in the control plane VCN 1216. The customer may desire to deploy or otherwise use resources in the data plane VCN 1218 that are provisioned in the control plane VCN 1216, and the data plane mirror app tier 1240 can facilitate the desired deployment, or other usage of resources, of the customer.

In some embodiments, the customer of the IaaS provider can apply filters to the data plane VCN 1218. In this embodiment, the customer can determine what the data plane VCN 1218 can access, and the customer may restrict access to public Internet 1254 from the data plane VCN 1218. The IaaS provider may not be able to apply filters or otherwise control access of the data plane VCN 1218 to any outside networks or databases. Applying filters and controls by the customer onto the data plane VCN 1218, contained in the customer tenancy 1221, can help isolate the data plane VCN 1218 from other customers and from public Internet 1254.

In some embodiments, cloud services 1256 can be called by the service gateway 1236 to access services that may not exist on public Internet 1254, on the control plane VCN 1216, or on the data plane VCN 1218. The connection between cloud services 1256 and the control plane VCN 1216 or the data plane VCN 1218 may not be live or continuous. Cloud services 1256 may exist on a different network owned or operated by the IaaS provider. Cloud services 1256 may be configured to receive calls from the service gateway 1236 and may be configured to not receive calls from public Internet 1254. Some cloud services 1256 may be isolated from other cloud services 1256, and the control plane VCN 1216 may be isolated from cloud services 1256 that may not be in the same region as the control plane VCN 1216. For example, the control plane VCN 1216 may be located in “Region 1,” and cloud service “Deployment 8,” may be located in Region 1 and in “Region 2.” If a call to Deployment 8 is made by the service gateway 1236 contained in the control plane VCN 1216 located in Region 1, the call may be transmitted to Deployment 8 in Region 1. In this example, the control plane VCN 1216, or Deployment 8 in Region 1, may not be communicatively coupled to, or otherwise in communication with, Deployment 8 in Region 2.

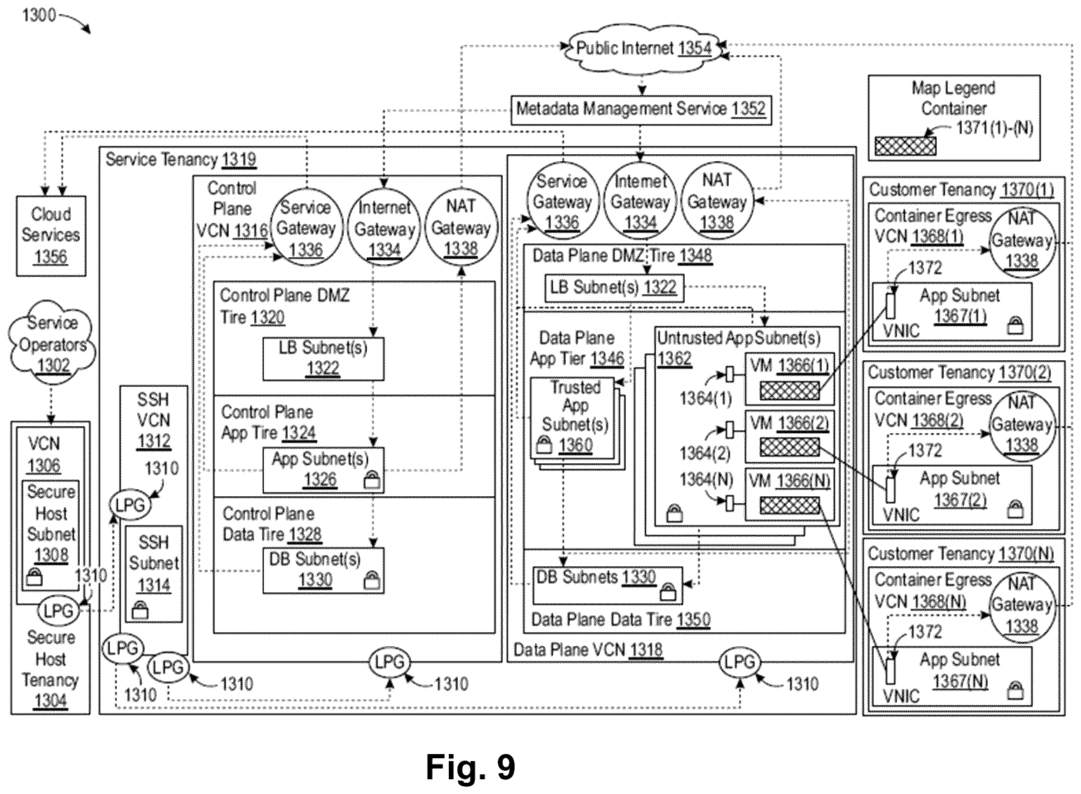

FIG. 9 is a block diagram 1300 illustrating another example pattern of an IaaS architecture, according to at least one embodiment. Service operators 1302 (e.g., service operators 1102) can be communicatively coupled to a secure host tenancy 1304 (e.g., the secure host tenancy 1104) that can include a virtual cloud network (VCN) 1306 (e.g., the VCN 1106) and a secure host subnet 1308 (e.g., the secure host subnet 1108). The VCN 1306 can include an LPG 1310 (e.g., the LPG 1110) that can be communicatively coupled to an SSH VCN 1312 (e.g., the SSH VCN 1112) via an LPG 1310 contained in the SSH VCN 1312. The SSH VCN 1312 can include an SSH subnet 1314 (e.g., the SSH subnet 1114), and the SSH VCN 1312 can be communicatively coupled to a control plane VCN 1316 (e.g., the control plane VCN 1116) via an LPG 1310 contained in the control plane VCN 1316 and to a data plane VCN 1318 (e.g., the data plane 1118) via an LPG 1310 contained in the data plane VCN 1318. The control plane VCN 1316 and the data plane VCN 1318 can be contained in a service tenancy 1319 (e.g., the service tenancy 1119).

The control plane VCN 1316 can include a control plane DMZ tier 1320 (e.g. the control plane DMZ tier 1120) that can include load balancer (“LB”) subnet(s) 1322 (e.g., LB subnet(s) 1122), a control plane app tier 1324 (e.g., the control plane app tier 1124) that can include app subnet(s) 1326 (e.g., similar to app subnet(s) 1126), a control plane data tier 1328 (e.g. the control plane data tier 1128) that can include DB subnet(s) 1330. The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1134) that can be contained in the control plane VCN 1316, and the app subnet(s) 1326 can be communicatively coupled to the DB subnet(s) 1330 contained in the control plane data tier 1328 and to a service gateway 1336 (e.g., the service gateway) and a network address translation (NAT) gateway 1338 (e.g., the NAT gateway 1138). The control plane VCN 1316 can include the service gateway 1336 and the NAT gateway 1338.

The data plane VCN 1318 can include a data plane app tier 1346 (e.g. the data plane app tier 1146), a data plane DMZ tier 1348 (e.g., the data plane DMZ tier 1148), and a data plane data tier 1350 (e.g., the data plane data tier 1150). The data plane DMZ tier 1348 can include LB subnet(s) 1322 that can be communicatively coupled to trusted app subnet(s) 1360 and untrusted app subnet(s) 1362 of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include one or more primary VNICs 1364(1)-(N) that can be communicatively coupled to tenant virtual machines (VMs) 1366(1)-(N). Each tenant VM 1366(1)-(N) can be communicatively coupled to a respective app subnet 1367(1)-(N) that can be contained in respective container egress VCNs 1368(1)-(N) that can be contained in respective customer tenancies 1370(1)-(N). Respective secondary VNICs 1372(1)-(N) can facilitate communication between the untrusted app subnet(s) 1362 contained in the data plane VCN 1318 and the app subnet contained in the container egress VCNs 1368(1)-(N). Each container egress VCNs 1368(1)-(N) can include a NAT gateway 1338 that can be communicatively coupled to public Internet 1354 (e.g. public Internet 1154).

The Internet gateway 1334 contained in the control plane VCN 1316 and contained in the data plane VCN 1318 can be communicatively coupled to a metadata management service 1352 (e.g. the metadata management system 1152) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively couple to cloud services 1356.

In some embodiments, the data plane VCN 1318 can be integrated with customer tenancies 1370. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane tier app 1346. Code to run the function may be executed in the VMs 1366(1)-(N), and the code may not be configured to run anywhere else on the data plane VCN 1318. Each VM 1366(1)-(N) may be connected to one customer tenancy 1370. Respective containers 1371(1)-(N) contained in the VMs 1366(1)-(N) may be configured to run the code. In this case, there can be a dual isolation (e.g., the containers 1371(1)-(N) running code, where the containers 1371(1)-(N) may be contained in at least the VM 1366(1)-(N) that are contained in the untrusted app subnet(s) 1362), which may help prevent incorrect or otherwise undesirable code from damaging the network of the IaaS provider or from damaging a network of a different customer. The containers 1371(1)-(N) may be communicatively coupled to the customer tenancy 1370 and may be configured to transmit or receive data from the customer tenancy 1370. The containers 1371(1)-(N) may not be configured to transmit or receive data from any other entity in the data plane VCN 1318. Upon completion of running the code, the IaaS provider may kill or otherwise dispose of the containers 1371(1)-(N).

In some embodiments, the trusted app subnet(s) 1360 may run code that may be owned or operated by the IaaS provider. In this embodiment, the trusted app subnet(s) 1360 may be communicatively coupled to the DB subnet(s) 1330 and be configured to execute CRUD operations in the DB subnet(s) 1330. The untrusted app subnet(s) 1362 may be communicatively coupled to the DB subnet(s) 1330, but in this embodiment, the untrusted app subnet(s) may be configured to execute read operations in the DB subnet(s) 1330. The containers 1371(1)-(N) that can be contained in the VM 1366(1)-(N) of each customer and that may run code from the customer may not be communicatively coupled with the DB subnet(s) 1330.

In other embodiments, the control plane VCN 1316 and the data plane VCN 1318 may not be directly communicatively coupled. In this embodiment, there may be no direct communication between the control plane VCN 1316 and the data plane VCN 1318. However, communication can occur indirectly through at least one method. An LPG 1310 may be established by the IaaS provider that can facilitate communication between the control plane VCN 1316 and the data plane VCN 1318. In another example, the control plane VCN 1316 or the data plane VCN 1318 can make a call to cloud services 1356 via the service gateway 1336. For example, a call to cloud services 1356 from the control plane VCN 1316 can include a request for a service that can communicate with the data plane VCN 1318.

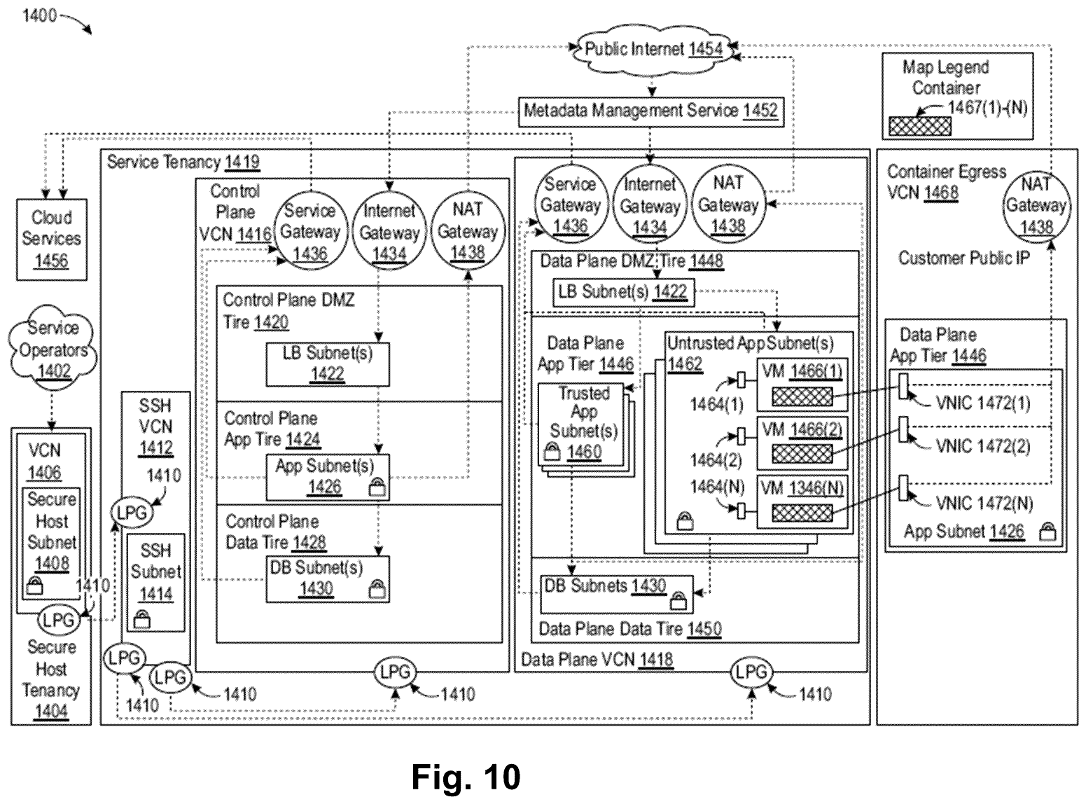

FIG. 10 is a block diagram 1400 illustrating another example pattern of an IaaS architecture, according to at least one embodiment. Service operators 1402 (e.g., service operators 1102) can be communicatively coupled to a secure host tenancy 1404 (e.g., the secure host tenancy 1104) that can include a virtual cloud network (“VCN”) 1406 (e.g., the VCN 1106) and a secure host subnet 1408 (e.g. the secure host subnet 1108). The VCN 1406 can include an LPG 1410 (e.g., the LPG 1110) that can be communicatively coupled to an SSH VCN 1412 (e.g., the SSH VCN 1112) via an LPG 1410 contained in the SSH VCN 1412. The SSH VCN 1412 can include an SSH subnet 1414 (e.g. the SSH subnet 1114), and the SSH VCN 1412 can be communicatively coupled to a control plane VCN 1416 (e.g., the control plane VCN 1116) via an LPG 1410 contained in the control plane VCN 1416 and to a data plane VCN 1418 (e.g., the data plane 1118) via an LPG 1410 contained in the data plane VCN 1418. The control plane VCN 1416 and the data plane VCN 1418 can be contained in a service tenancy 1419 (e.g., the service tenancy 1119).

The control plane VCN 1416 can include a control plane DMZ tier 1420 (e.g., the control plane DMZ tier 1120) that can include LB subnet(s) 1422 (e.g. LB subnet(s) 1122), a control plane app tier 1424 (e.g., the control plane app tier 1124) that can include app subnet(s) 1426 (e.g., app subnet(s) 1126), a control plane data tier 1428 (e.g., the control plane data tier 1128) that can include DB subnet(s) 1430 (e.g., DB subnet(s) 1330). The LB subnet(s) 1422 contained in the control plane DMZ tier 1420 can be communicatively coupled to the app subnet(s) 1426 contained in the control plane app tier 1424 and to an Internet gateway 1434 (e.g., the Internet gateway 1134) that can be contained in the control plane VCN 1416, and the app subnet(s) 1426 can be communicatively coupled to the DB subnet(s) 1430 contained in the control plane data tier 1428 and to a service gateway 1436 (e.g., the service gateway of FIG. 5) and a network address translation (NAT) gateway 1438 (e.g., the NAT gateway 1138). The control plane VCN 1416 can include the service gateway 1436 and the NAT gateway 1438.

The data plane VCN 1418 can include a data plane app tier 1446 (e.g. the data plane app tier 1146), a data plane DMZ tier 1448 (e.g. the data plane DMZ tier 1148), and a data plane data tier 1450 (e.g. the data plane data tier 1150). The data plane DMZ tier 1448 can include LB subnet(s) 1422 that can be communicatively coupled to trusted app subnet(s) 1460 (e.g. trusted app subnet(s) 1360) and untrusted app subnet(s) 1462 (e.g. untrusted app subnet(s) 1362) of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include primary VNICs 1464(1)-(N) that can be communicatively coupled to tenant virtual machines (VMs) 1466(1)-(N) residing within the untrusted app subnet(s) 1462. Each tenant VM 1466(1)-(N) can run code in a respective container 1467(1)-(N), and be communicatively coupled to an app subnet 1426 that can be contained in a data plane app tier 1446 that can be contained in a container egress VCN 1468. Respective secondary VNICs 1472(1)-(N) can facilitate communication between the untrusted app subnet(s) 1462 contained in the data plane VCN 1418 and the app subnet contained in the container egress VCN 1468. The container egress VCN can include a NAT gateway 1438 that can be communicatively coupled to public Internet 1454 (e.g. public Internet 1154).

The Internet gateway 1434 contained in the control plane VCN 1416 and contained in the data plane VCN 1418 can be communicatively coupled to a metadata management service 1452 (e.g. the metadata management system 1152) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively couple to cloud services 1456.

In some examples, the pattern illustrated by the architecture of block diagram 1400 may be considered an exception to the pattern illustrated by the architecture of block diagram 1300 and may be desirable for a customer of the IaaS provider if the IaaS provider cannot directly communicate with the customer (e.g., a disconnected region). The respective containers 1467(1)-(N) that are contained in the VMs 1466(1)-(N) for each customer can be accessed in real-time by the customer. The containers 1467(1)-(N) may be configured to make calls to respective secondary VNICs 1472(1)-(N) contained in app subnet(s) 1426 of the data plane app tier 1446 that can be contained in the container egress VCN 1468. The secondary VNICs 1472(1)-(N) can transmit the calls to the NAT gateway 1438 that may transmit the calls to public Internet 1454. In this example, the containers 1467(1)-(N) that can be accessed in real-time by the customer can be isolated from the control plane VCN 1416 and can be isolated from other entities contained in the data plane VCN 1418. The containers 1467(1)-(N) may also be isolated from resources from other customers.

In other examples, the customer can use the containers 1467(1)-(N) to call cloud services 1456. In this example, the customer may run code in the containers 1467(1)-(N) that requests a service from cloud services 1456. The containers 1467(1)-(N) can transmit this request to the secondary VNICs 1472(1)-(N) that can transmit the request to the NAT gateway that can transmit the request to public Internet 1454. Public Internet 1454 can transmit the request to LB subnet(s) 1422 contained in the control plane VCN 1416 via the Internet gateway 1434. In response to determining the request is valid, the LB subnet(s) can transmit the request to app subnet(s) 1426 that can transmit the request to cloud services 1456 via the service gateway 1436.

It should be appreciated that IaaS architectures 1100, 1200, 1300, 1400 depicted in the figures may have other components than those depicted. Further, the embodiments shown in the figures are only some examples of a cloud infrastructure system that may incorporate certain embodiments. In some other embodiments, the IaaS systems may have more or fewer components than shown in the figures, may combine two or more components, or may have a different configuration or arrangement of components.

As disclosed, embodiments gather data from a wide range of sources including medical and non-medical, and analyze the data with AI with the goal of improving the competitiveness of physicians which in turn leads to better patient outcomes. Embodiments implement a trained generative AI model to generate a unique and novel set of insights/recommendations in response to a wide variety of input data

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of providing healthcare information to a plurality of healthcare providers, the method comprising:
receiving first historical information corresponding to the plurality of healthcare providers;
receiving second historical information corresponding to external health care information sources;
training an artificial intelligence (AI) model using the first historical information and the second historical information;
receiving current information corresponding to the plurality of healthcare providers and/or the external health care information sources; and
in response to the current information, generating one or more healthcare suggestions by the AI model and delivering the suggestions to one or more of the plurality of healthcare providers.

2. The method of claim 1, wherein the first historical information comprises one or more of: data on the healthcare providers; information related to healthcare personnel; patient demography; clinical information or patient outcomes.

3. The method of claim 1, wherein the second historical information comprises one or more of: latest therapeutic protocols and drugs; opportunities for continuing medical education; local conditions; disease outbreaks and accidents; or natural disasters and calamities.

4. The method of claim 1, wherein the suggestions comprise one or more of: latest approved protocols for treatment; new generation of drugs available; or contact information of other physicians who have treated similar conditions successfully.

5. The method of claim 1, wherein the suggestions comprise one or more of: details of available training; recommended associations of doctors to join; or research articles to review.

6. The method of claim 1, wherein the suggestions comprise one or more of:
an indication of drugs to immediately procure; latest information regarding a disease outbreak; or resources for emergency procedure training.

7. The method of claim 1, wherein the AI model comprises a generative AI model, further comprising:
retraining the AI model in response to actions taken by the plurality of healthcare providers in response to the suggestions.

8. The method of claim 1, further comprising:
using a cloud infrastructure for providing healthcare information, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.

9. A computer readable medium having instructions stored thereon that, when executed by one or more processors, cause the processors to provide healthcare information to a plurality of healthcare providers, the providing healthcare information comprising:
receiving first historical information corresponding to the plurality of healthcare providers;
receiving second historical information corresponding to external health care information sources;
training an artificial intelligence (AI) model using the first historical information and the second historical information;
receiving current information corresponding to the plurality of healthcare providers and/or the external health care information sources; and
in response to the current information, generating one or more healthcare suggestions by the AI model and delivering the suggestions to one or more of the plurality of healthcare providers.

10. The computer readable medium of claim 9, wherein the first historical information comprises one or more of: data on the healthcare providers; information related to healthcare personnel; patient demography; clinical information or patient outcomes.

11. The computer readable medium of claim 9, wherein the second historical information comprises one or more of: latest therapeutic protocols and drugs; opportunities for continuing medical education; local conditions; disease outbreaks and accidents; or natural disasters and calamities.

12. The computer readable medium of claim 9, wherein the suggestions comprise one or more of: latest approved protocols for treatment; new generation of drugs available; or contact information of other physicians who have treated similar conditions successfully.

13. The computer readable medium of claim 9, wherein the suggestions comprise one or more of: details of available training; recommended associations of doctors to join; or research articles to review.

14. The computer readable medium of claim 9, wherein the suggestions comprise one or more of:
an indication of drugs to immediately procure; latest information regarding a disease outbreak; or resources for emergency procedure training.

15. The computer readable medium of claim 9, wherein the AI model comprises a generative AI model, the providing healthcare information further comprising:
retraining the AI model in response to actions taken by the plurality of healthcare providers in response to the suggestions.

16. The method of claim 1, the providing healthcare information further comprising:
using a cloud infrastructure for providing healthcare information, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.

17. A cloud based system for providing healthcare information to a plurality of healthcare providers, the system comprising:
an artificial intelligence (AI) model;
one or more processors coupled to the AI model and configured to:
receive first historical information corresponding to the plurality of healthcare providers;
receive second historical information corresponding to external health care information sources;
train the AI model using the first historical information and the second historical information;
receive current information corresponding to the plurality of healthcare providers and/or the external health care information sources; and
in response to the current information, generate one or more healthcare suggestions by the AI model and delivering the suggestions to one or more of the plurality of healthcare providers.

18. The cloud based system of claim 17, wherein the first historical information comprises one or more of: data on the healthcare providers; information related to healthcare personnel; patient demography; clinical information or patient outcomes.

19. The cloud based system of claim 17, wherein the second historical information comprises one or more of: latest therapeutic protocols and drugs; opportunities for continuing medical education; local conditions; disease outbreaks and accidents; or natural disasters and calamities.

20. The cloud based system of claim 17, wherein the system is executed on a cloud infrastructure, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.
